(19) United States
(12) Patent Application Publication
(10) Pub. No.: US 2021/0307658 A1
(43) Pub. Date: Oct. 7, 2021

(54) Glucose Annunciator And Method Of Operation Thereof

(71) Applicant: Stewart Financial Holdings, Inc., Allen, TX (US)

(72) Inventors: William J. STEWART, College Station, TX (US); Achu Geetha BYJU, College Station, TX (US)

(21) Appl. No.: 17/216,677

(22) Filed: Mar. 30, 2021

Related U.S. Application Data
(60) Provisional application No. 63/004,349, filed on Apr. 2, 2020.

(51) Int. Cl. A61B 5/145; A61B 5/00

(52) U.S. Cl. CPC A61B 5/14532; A61B 5/0004; A61B 5/0022; A61B 5/7455; A61B 5/7405; A61B 5/742; A61B 5/746

(57) ABSTRACT

A glucose annunciator comprising: i) a data eavesdropping interface configured to acquire blood glucose data transmitted by a wireless glucose monitor, the blood glucose data indicating a blood glucose level of a subject; and ii) an alert generator coupled to the data eavesdropping interface and configured to generate at least one of an audible indicium, a visible indicium, or a haptic indicium based on the blood glucose data but remote from the wireless glucose monitor. The indicium at least generally indicates the blood glucose level of the subject without requiring access to the wireless glucose monitor.

### RELATED APPLICATIONS

The present application is related to U.S. Provisional Patent No. 63/004,349, filed Apr. 2, 2020, entitled “Glucose Annunciator And Method Of Operation Thereof”. Provisional Patent No. 63/004,349 is assigned to the assignee of the present application and is hereby incorporated by reference into the present application as if fully set forth herein. The present application hereby claims priority under 35 U.S.C. § 119(e) to U.S. Provisional Patent No. 63/004,349.

### TECHNICAL FIELD

This application is directed, in general, to warning devices and, more specifically, to a glucose annunciator capable of warning when a subject's blood glucose level is approaching an unsafe level.

### BACKGROUND

Diabetes is a serious problem not only in the United States, but worldwide. One of the central issues a diabetic faces is managing blood glucose levels. Hyperglycemia (abnormally high blood glucose levels) damages nerves, blood vessels, and organs and can lead to ketoacidosis or hyperglycemic hyperosmolar nonketotic syndrome (HHNS), depending upon whether the diabetes is Type I (abnormally low insulin production) or Type II (abnormally high insulin resistance). Hypoglycemia (abnormally low blood glucose levels) is more acute, beginning with confusion, dizziness, and weakness, and potentially leading to unconsciousness, coma, and death. The diabetic's ongoing pursuit is to keep blood glucose levels within a range of acceptable levels, avoiding foods and eating patterns that would cause blood glucose levels to depart from the physiologically safe range, taking insulin as needed, and ceasing activities that would present a danger to the person when blood glucose levels are too low.

Unfortunately, until the last 50 years or so, diabetics were relegated to submitting blood samples to a remote laboratory for analysis, which is a slow and expensive process. Diabetics were resigned to doing their best to avoid dangerous blood glucose levels or activities that would present a risk to themselves or others.

The market responded with various tools to equip diabetics to measure blood glucose levels as they desired and without having to involve a laboratory. First to arrive were test kits with which a blood sample is drawn and brought into contact with a chemical that responds by changing color to indicate the blood glucose level. Later came electronic glucose meters (also known as glucometers, glucomonitors, blood glucose meters, glucose monitors or diabetes monitors) that analyze electrical or optical characteristics of a blood or interstitial fluid sample to determine its glucose concentration. The latest technology includes minimally invasive, noninvasive, or implantable electronic wireless glucose monitors that provide glucose data to a smartphone, allowing test results to be aggregated over time for analysis not only by the diabetic but also by specialized pattern analysis equipment and healthcare professionals. Some of these monitors provide a continuous stream of test results, lending them their name: continuous glucose monitors (CGMs). These tools have allowed diabetics to take control of their disease, extending not only their lifespan, but also their quality of life.

### SUMMARY

It is an aspect of the present disclosure to provide a glucose annunciator comprising: i) a data eavesdropping interface configured to acquire blood glucose data transmitted by a wireless glucose monitor, the blood glucose data indicating a blood glucose level of a subject; and ii) an alert generator coupled to the data eavesdropping interface and configured to generate at least one of an audible indicium, a visible indicium, or a haptic indicium based on the blood glucose data but remote from the wireless glucose monitor. The indicium at least generally indicates the blood glucose level of the subject without requiring access to the wireless glucose monitor.

In one embodiment, the wireless glucose monitor is configured to transmit the blood glucose data to a smartphone for further processing by a monitor application executing thereon, the data eavesdropping interface configured to acquire the blood glucose data without interfering in an operation of the wireless glucose monitor or the monitor application.

In another embodiment, the data eavesdropping interface is configured to acquire the blood glucose data by eavesdropping on a wireless transmission of the blood glucose data.

In still another embodiment, the data eavesdropping interface is configured to acquire the blood glucose data by reading the blood glucose data in a smartphone associated with the wireless glucose monitor.

In yet another embodiment, the audible indicium is based on the blood glucose data by having at least one of: i) a pitch based on the blood glucose data, ii) a volume based on the blood glucose data, and iii) an intermittence based on the blood glucose data.

In a further embodiment, the visible indicium is based on the blood glucose data by having at least one of: i) a wavelength based on the blood glucose data, ii) an intensity based on the blood glucose data, and iii) an intermittence based on the blood glucose data.

In a still further embodiment, the annunciator further comprises an ambient light sensor configured to provide a signal indicating an ambient light level, an intensity of the visible indicium being at least partially based on the ambient light level.

It is another aspect of the present disclosure to provide a method of announcing blood glucose level comprising: i) eavesdropping on blood glucose data transmitted by a wireless glucose monitor, the blood glucose data indicating a blood glucose level of a subject; and ii) generating at least one of an audible indicium, a visible indicium, or a haptic indicium based on the blood glucose data but remote from the wireless glucose monitor, the indicium at least generally indicating the blood glucose level of the subject without requiring access to the wireless glucose monitor.

In one embodiment, the wireless glucose monitor is configured to transmit the blood glucose data to a smartphone for further processing by a monitor application executing thereon, the eavesdropping comprising eavesdropping on the blood glucose data without interfering in an operation of the wireless glucose monitor or the monitor application.

In another embodiment, the eavesdropping comprises intercepting a wireless transmission of the blood glucose data.

In still another embodiment, the eavesdropping comprises reading the blood glucose data in a smartphone associated with the wireless glucose monitor.

In yet another embodiment, the audible indicium is based on the blood glucose data by having at least one of: i) a pitch based on the blood glucose data, ii) a volume based on the blood glucose data, and iii) an intermittence based on the blood glucose data.

In a further embodiment, the visible indicium is based on the blood glucose data by having at least one of: i) a wavelength based on the blood glucose data, ii) an intensity based on the blood glucose data, and iii) an intermittence based on the blood glucose data.

In a still further embodiment, the method further comprises basing an intensity of the visible indicium at least partially based on an ambient light level.

It is still another aspect of the present disclosure to provide a glucose annunciator for use with a wireless glucose monitor configured to transmit blood glucose data indicating a blood glucose level of a subject to a smartphone for further processing by a monitor application executing thereon. The glucose annunciator comprises: i) a data eavesdropping interface configured to acquire the blood glucose data without interfering in an operation of the wireless glucose monitor or the monitor application; and ii) an alert generator coupled to the data eavesdropping interface and configured to generate at least one of an audible indicium, a visible indicium, or a haptic indicium based on the blood glucose data but remote from the wireless glucose monitor, the indicium indicating when the blood glucose level of the subject is approaching an unsafe level without requiring access to the wireless glucose monitor.

In one embodiment, the data eavesdropping interface is configured to acquire the blood glucose data by intercepting a wireless transmission of the blood glucose data.

In another embodiment, the data eavesdropping interface is configured to acquire the blood glucose data by reading the blood glucose data in a smartphone associated with the wireless glucose monitor.

In still another embodiment, wherein the audible indicium is based on the blood glucose data by having at least one of: i) a pitch based on the blood glucose data, ii) a volume based on the blood glucose data, and iii) an intermittence based on the blood glucose data.

In yet another embodiment, the visible indicium is based on the blood glucose data by having at least one of: i) a wavelength based on the blood glucose data, ii) an intensity based on the blood glucose data, and iii) an intermittence based on the blood glucose data.

In a further embodiment, the annunciator further comprises an ambient light sensor configured to provide a signal indicating an ambient light level, an intensity of the visible indicium being at least partially based on the ambient light level.

### BRIEF DESCRIPTION

Reference is now made to the following descriptions taken in conjunction with the accompanying drawings, in which:

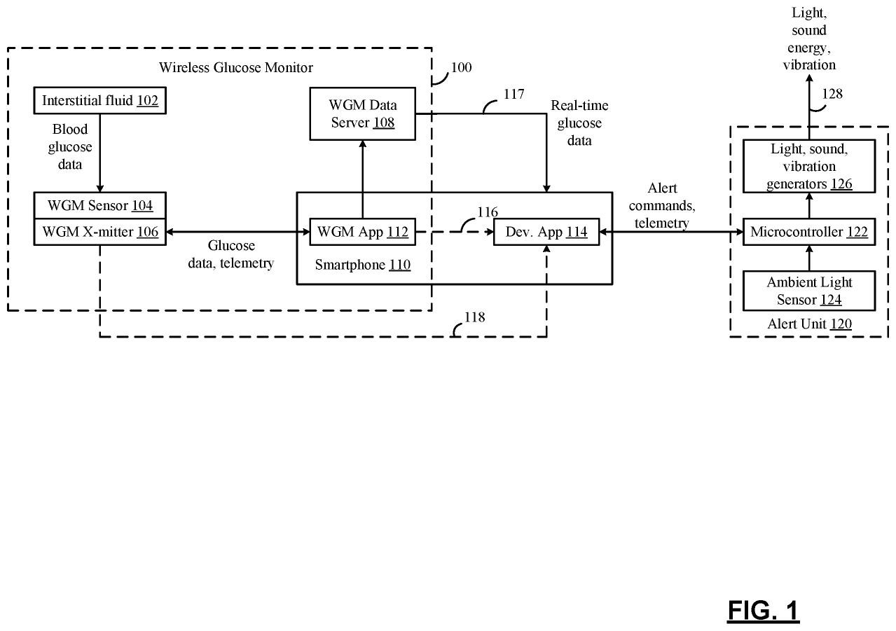

FIG. 1 is a block diagram of one embodiment of a glucose annunciator in the environment of a wireless glucose monitor (WGM) and a smartphone; and

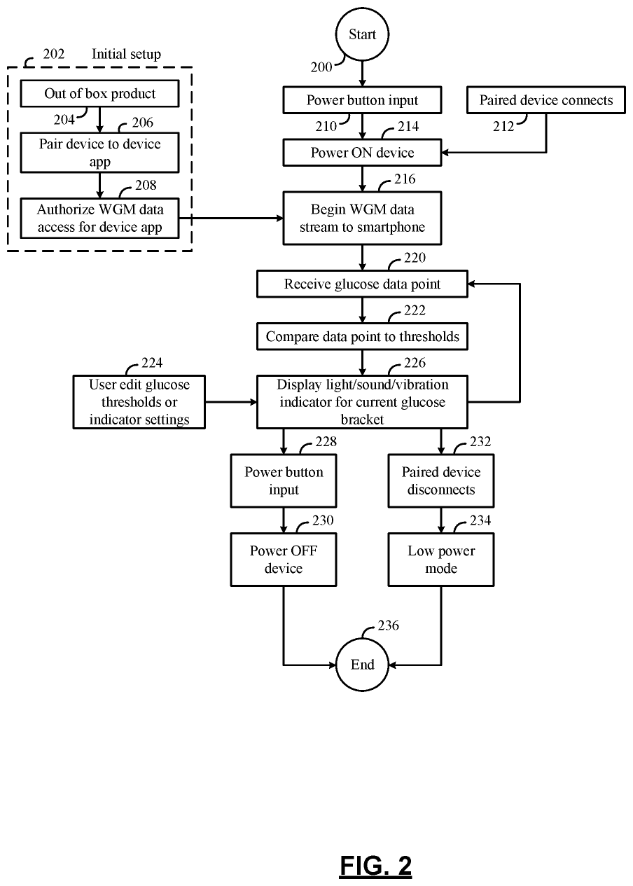

FIG. 2 is a flow diagram of one embodiment of a method of announcing glucose level.

### DETAILED DESCRIPTION

As described above, tools such as electronic glucose meters, including minimally invasive, noninvasive or implantable electronic monitors, continuous or otherwise, have allowed diabetics to take control of their disease, extending their lifespan and improving their quality of life. However, these monitors still require significant interaction by the individual to read the blood glucose data. Unfortunately, diabetics sometimes find themselves in situations when reading a glucose meter is inconvenient, or even dangerous. One notable example is while driving a vehicle. An individual's blood glucose level sometimes not only presents a risk to the diabetic, but third parties as well, for example, passengers in a vehicle a diabetic is driving. A diabetic may be less attentive to their glucose while driving which can lead to a hypoglycemic event. Falling unconscious as a result of this event while behind the wheel of a motor vehicle could lead to severe harm to the diabetic driver, as well as passengers or other bystanders. Not only the diabetic, but also these third parties have a stake in the diabetic's health.

Introduced herein are various embodiments of a glucose annunciator and a method of announcing glucose level. The annunciator is not a glucose monitor, and the method is not a method of detecting blood glucose or determining blood glucose levels. The annunciator and method embodiments do not detect or determine blood glucose levels. Instead, the annunciator and method work in conjunction with various commercially available electronic glucose monitors, eavesdropping on glucose data a monitor has detected or determined and wirelessly transmitted. The annunciator and method transform the glucose data into one or both of audible and visible indicia that allow the glucose level of a diabetic (also referred to herein as a “subject”) to be more prominently known without requiring access to the monitor.

“Eavesdropping,” as that term is used herein, is defined as acquiring communicated or stored data without interfering in the transmission or reception of communicated data or the storage or retrieval of the stored data. Eavesdropping therefore encompasses acquiring data from a wireline or wireless data transmission without interfering in the operation of the transmitter transmitting the data or the receiver normally intended to receive that data. Eavesdropping also encompasses reading data from a memory or data storage unit without interfering with write or read operations performed with respect to that memory or data storage unit. Eavesdropping also encompasses reading data as it is temporarily communicated or stored within an application, or app, without substantially interfering in the operation or execution of the app.

One apparent application of the glucose annunciator is to place it in a conspicuous place in a motor vehicle and use it to alert the driver and passengers to an unsafe glucose level, which would be particularly germane if the subject is the driver. Another application is to place the annunciator in a room (e.g., a bedroom) of a home to alert its occupants of an unsafe glucose level, perhaps at night. It may also be used in an office or hospital setting, or even outdoors, such as while camping, as desired.

“Access,” as that term is used herein, is defined as the ability to (1) read a display on the monitor, (2) see a warning indicator on the monitor, (3) hear a warning sound produced by the monitor, or (4) feel a warning vibration produced by the monitor. Thus, the glucose annunciator provides insight regarding glucose level to persons who are outside the range of the monitor itself, as well as insight that is more obvious to the diabetic (subject). The glucose annunciator preferably functions without interfering in the operation of the monitor. The glucose annunciator and method then generate one or more of an audible indicium (i.e., one or more sounds), a visible indicium (e.g., a light of a particular color or color pattern), or a haptic indicium (e.g., a vibration) that a diabetic or a third party may hear, see, or feel to inform them of the blood glucose level.

The one or more indicia mean different things, depending upon the embodiment. In one embodiment, the glucose annunciator and method generate the one or more indicia only when blood glucose levels lie outside of a range of levels generally considered as being safe. The indicia, when present, therefore represent either hyperglycemia or hypoglycemia. For example, a red light may indicate either high or low blood glucose. In an alternative embodiment, the glucose annunciator and method generate the one or more indicia only when blood glucose levels fall below a threshold level generally considered as being safe. The indicia, when present, therefore represents hypoglycemia.

For example, a flashing red light may indicate low blood glucose. In another alternative embodiment, the glucose annunciator and method generate the one or more indicia constantly, varying the types or qualities of indicia depending upon whether blood glucose levels lie inside or outside of the range of levels generally considered as being safe. For example, a green or blue light and no sound may indicate that blood glucose levels are normal, while a yellow light and a sound may indicate high blood glucose levels and a red light and a louder or a more disturbing or importunate sound may indicate low blood glucose levels.

The audible and visible indicia may vary in different ways to indicate different blood glucose level or whether the level is above or below a threshold or whether or not the blood glucose level is within the safe range, depending upon the embodiment. In various embodiments, the audible indicium may change in terms of pitch, volume, or intermittence (e.g., on/off rate or change in volume). In various embodiments, the visible indicium may change in terms of wavelength, intensity, or intermittence.

The glucose annunciator acquires the blood glucose data transmitted by the WGM in different ways, depending upon the embodiment. In one embodiment, the glucose annunciator is configured to eavesdrop on wireless transmission of blood glucose data emanating from the wireless glucose meter. In another embodiment, the WGM is designed to operate with a monitor application executing on a smartphone, whereby the monitor application stores the blood glucose data in the smartphone, in which case the glucose annunciator is configured to acquire the blood glucose data by reading the memory of the smartphone.

In some embodiments, the glucose annunciator includes an ambient light sensor configured to provide a signal indicating an ambient light level, which allows the glucose annunciator to vary the intensity of any visible indicium it may generate based on the ambient light level. This feedback allows the visible indicium to be perceived in daylight without being overwhelmingly bright in the dark.

Particular embodiments will now be described with reference to particular figures. FIG. 1 is a block diagram of one embodiment of a glucose annunciator in the environment of a WGM and a smartphone. A broken line 100 surrounds elements considered to be parts and work pieces of a WGM.

A WGM sensor 104 comes into contact or communication with interstitial fluid (e.g., blood or fluid containing blood) 102 of a subject (i.e., a diabetic), allowing the WGM sensor 104 to detect one or properties of the interstitial fluid 102 that are indicative of blood glucose level. The WGM sensor 104 may, but need not, determine the blood glucose level from the one or more properties. A WGM transmitter 106 coupled to the WGM sensor 104 transmits blood glucose data bearing the one or more properties or the determined blood glucose level to a WGM application (or “app”) 112 executing in a smartphone 110. In one embodiment, the wireless transmission is made according to Bluetooth®, ZigBee®, infrared light or another relatively short-range wireless convention. In the embodiment of FIG. 1, the smartphone is a commercially available smartphone such as an iPhone® commercially available from Apple Inc. of Cupertino, Calif., or a smartphone under control of an Android® operating system, such as is commercially available from Samsung of Seoul, South Korea.

If the blood glucose data transmitted by the WGM transmitter 106 bears the one or more properties, the app 112 determines the blood glucose level from the one or more properties. If the blood glucose data transmitted by the WGM transmitter 106 bears the blood glucose level as determined by the WGM sensor 104, the WGM app 112 does not need to determine the blood glucose level.

The WGM sensor 104 may be coupled to a display (which is often a low-power liquid crystal display (LCD)) and may be coupled to circuitry for providing a sound, a light or vibration. As those familiar with WGMs understand, the sound, light, or vibration, to the extent provided, are of relatively small power and effect and designed to warn only the subject of a dangerous blood glucose level.

In either case, the WGM app 112 stores the blood glucose level in a database (not shown) located in a memory (not shown) of the smartphone 110 and causes the blood glucose level to be displayed on a display (not shown) of the smartphone 110. The WGM app 112 may further transmit the blood glucose level, via the Internet (not shown), to a WGM data server 108 for further storage and analysis.

As described above, the glucose annunciator includes a data eavesdropping interface and an alert generator. The glucose annunciator embodiment of FIG. 1 takes the form of a device app 114 configured to execute in the smartphone 110 and an alert unit 120 wirelessly communicates with the device app 114 through the smartphone. In general, the data eavesdropping interface is configured to acquire blood glucose data transmitted by the WGM, wherein the blood glucose data indicates the subject's blood glucose level. In the embodiment of FIG. 1, the data eavesdropping interface is embodied as a sequence of computer instructions executable in a general-purpose processor and is part of the device app 114. In various embodiments, the device app 114 acquires blood glucose data emanating from the WCM either by reading the blood glucose data from the database in the memory of the smartphone 110 (as a broken line 116 signifies), by extracting the blood glucose data from the WGM data server (as a solid line 117 signifies), or by eavesdropping on wireless transmissions of the blood glucose data emanating from the WGM transmitter 106 (as a broken line 118 signifies).

The alert generator is coupled to the data eavesdropping interface and generates at least one of an audible indicium, a visible indicium, or a haptic indicium based on the blood glucose data but remote from the WGM, wherein the indicium at least generally indicates the subject's blood glucose level without requiring access to the monitor itself. In the embodiment of FIG. 1, the alert generator comprises the device app 114 and the alert unit 120. The portion of the alert generator that is embodied in the device app 114 is likewise embodied in a sequence of computer instructions executable in a general-purpose processor. The portion of the alert generator that is embodied in the alert unit 120 is a combination of software and hardware, namely: (1) a sequence of computer instructions executable in a microcontroller 122 of the alert unit 120, and (2) the microcontroller 122, an ambient light sensor 124 and light and sound generators 126.

In the embodiment of FIG. 1, the device app 114 receives blood glucose data and compares the blood glucose data relative to one or more ranges or thresholds indicating safe levels, hyperglycemia, hypoglycemia, and perhaps the extent to which the glucose data represents mild or serious hyperglycemia or hypoglycemia. The range(s) or threshold(s) may be “factory” defaults or user-determined at least to some extent. They may be arranged in a lookup table, so blood glucose levels “map” to particular indicia.

Based on this comparison, the device app 114 selects one or more of an audible indicium, a visible indicium (e.g., a pitch or volume of sound or color or intensity of light), or a haptic indicium and causes data indicative of the one or both to be wirelessly transmitted to the microcontroller 122. In one embodiment, the wireless transmission is made according to Bluetooth®, ZigBee®, infrared light or another relatively short-range wireless convention, preferably one the smartphone already has the hardware and software driver to accommodate. In an alternative embodiment, the device app 114 wirelessly transmits the glucose data itself to the microcontroller 122, which then carries out the range/threshold comparison and indicia selection.

Irrespective of where the comparison and selection are carried out, the light and sound generators 126 are enabled to generate the appropriate indicium or indicia, resulting in the emanation of light or sound energy, as an arrow 128 signifies. The intensity of any visible indicium may be changed in response to input provided by an ambient light sensor 124. Thus, any visible indicium may be balanced relative to ambient light conditions.

Turning now to FIG. 2, illustrated is a flow diagram of one embodiment of a method of announcing blood glucose level. The method begins in a start step 200. In an initial setup step sequence 202, a new glucose annunciator is initiated. Accordingly, in a step 204, the annunciator is removed from its box. A device app (116 of FIG. 1) is downloaded onto a smartphone, executed, and caused to pair with an alert unit (120 of FIG. 1) in a step 206. In a step 208, access to a WGM data server associated with an already-installed WGM app (112 of FIG. 1) is authorized. Given this permission, blood glucose data may be transferred directly from the WGM data server of FIG. 1, in accordance with the solid line 117. Absent this permission, blood glucose data is wirelessly eavesdropped on or read from the WGM database in the smartphone's memory. The glucose annunciator is now initialized.

Depending upon whether the glucose annunciator has been placed in a manual mode or automatic mode of operation, a power button of the glucose annunciator is manually pressed in a step 210, or the glucose annunciator automatically connects to its paired alert unit in a step 212. Either event triggers the alert unit to power on (enter a normal power mode) in a step 214.

In a step 216, blood glucose data begins to be received by the device app, either from the WGM data server 108, directly from the WGM transmitter 106 (if so authorized) or wirelessly eavesdropped on or read from smartphone memory (if not so authorized). In a step 220, the blood glucose data becomes sufficient to establish a blood glucose level. In a step 222, the blood glucose level is compared to one or more range(s) or threshold(s). In a step 226, corresponding audible, visible, and/or haptic indicia are selected and sounds, lights, and/or vibratory signals are generated in response. Steps 220, 222 and 226 repeat as further blood glucose data is received. Thus, the method encompasses continual eavesdropping and annunciating. In a step 224, the range(s), threshold(s) and audible and visible indicia properties may be edited.

In a step 228, if the glucose annunciator is operating in a manual mode, the power button may be pressed to turn power off to the alert unit. In a step 232, if the glucose annunciator is operating in an automatic mode, the alert unit enters a low-power mode (in a step 234) if its pairing with the smartphone is interrupted. The method ends in an end step 230.

Those skilled in the art to which this application relates will appreciate that other and further additions, deletions, substitutions, and modifications may be made to the described embodiments.

What is claimed is:

1. A glucose annunciator, comprising:
a data eavesdropping interface configured to acquire blood glucose data transmitted by a wireless glucose monitor, the blood glucose data indicating a blood glucose level of a subject; and
an alert generator coupled to the data eavesdropping interface and configured to generate at least one of an audible indicium, a visible indicium, or a haptic indicium based on the blood glucose data but remote from the wireless glucose monitor, the indicium at least generally indicating the blood glucose level of the subject without requiring access to the wireless glucose monitor.

2. The annunciator as recited in claim 1 wherein the wireless glucose monitor is configured to transmit the blood glucose data to a smartphone for further processing by a monitor application executing thereon, the data eavesdropping interface configured to acquire the blood glucose data without interfering in an operation of the wireless glucose monitor or the monitor application.

3. The annunciator as recited in claim 1 wherein the data eavesdropping interface is configured to acquire the blood glucose data by eavesdropping on a wireless transmission of the blood glucose data.

4. The annunciator as recited in claim 1 wherein the data eavesdropping interface is configured to acquire the blood glucose data by reading the blood glucose data in a smartphone associated with the wireless glucose monitor.

5. The annunciator as recited in claim 1 wherein the audible indicium is based on the blood glucose data by having at least one of:
a pitch based on the blood glucose data,
a volume based on the blood glucose data, and
an intermittence based on the blood glucose data.

6. The annunciator as recited in claim 1 wherein the visible indicium is based on the blood glucose data by having at least one of:
a wavelength based on the blood glucose data,
an intensity based on the blood glucose data, and
an intermittence based on the blood glucose data.

7. The annunciator as recited in claim 1 further comprising an ambient light sensor configured to provide a signal indicating an ambient light level, an intensity of the visible indicium being at least partially based on the ambient light level.

8. A method of announcing blood glucose level, comprising:
eavesdropping on blood glucose data transmitted by a wireless glucose monitor, the blood glucose data indicating a blood glucose level of a subject; and
generating at least one of an audible indicium, a visible indicium, or a haptic indicium based on the blood glucose data but remote from the wireless glucose monitor, the indicium at least generally indicating the blood glucose level of the subject without requiring access to the wireless glucose monitor.

9. The method as recited in claim 8 wherein the wireless glucose monitor is configured to transmit the blood glucose data to a smartphone for further processing by a monitor application executing thereon, the eavesdropping comprising eavesdropping on the blood glucose data without interfering in an operation of the wireless glucose monitor or the monitor application.

10. The method as recited in claim 8 wherein the eavesdropping comprises intercepting a wireless transmission of the blood glucose data.

11. The method as recited in claim 8 wherein the eavesdropping comprises reading the blood glucose data in a smartphone associated with the wireless glucose monitor.

12. The method as recited in claim 8 wherein the audible indicium is based on the blood glucose data by having at least one of:
a pitch based on the blood glucose data,
a volume based on the blood glucose data, and
an intermittence based on the blood glucose data.

13. The method as recited in claim 8 wherein the visible indicium is based on the blood glucose data by having at least one of:
a wavelength based on the blood glucose data,
an intensity based on the blood glucose data, and
an intermittence based on the blood glucose data.

14. The method as recited in claim 8 further comprising basing an intensity of the visible indicium at least partially based on an ambient light level.

15. For use with a wireless glucose monitor configured to transmit blood glucose data indicating a blood glucose level of a subject to a smartphone for further processing by a monitor application executing thereon, a glucose annunciator comprising:
a data eavesdropping interface configured to acquire the blood glucose data without interfering in an operation of the wireless glucose monitor or the monitor application; and
an alert generator coupled to the data eavesdropping interface and configured to generate at least one of an audible indicium, a visible indicium, or a haptic indicium based on the blood glucose data but remote from the wireless glucose monitor, the indicium indicating when the blood glucose level of the subject is approaching an unsafe level without requiring access to the wireless glucose monitor.

16. The annunciator as recited in claim 15 wherein the data eavesdropping interface is configured to acquire the blood glucose data by intercepting a wireless transmission of the blood glucose data.

17. The annunciator as recited in claim 15 wherein the data eavesdropping interface is configured to acquire the blood glucose data by reading the blood glucose data in a smartphone associated with the wireless glucose monitor.

18. The annunciator as recited in claim 15 wherein the audible indicium is based on the blood glucose data by having at least one of:
a pitch based on the blood glucose data,
a volume based on the blood glucose data, and
an intermittence based on the blood glucose data.

19. The annunciator as recited in claim 15 wherein the visible indicium is based on the blood glucose data by having at least one of:
a wavelength based on the blood glucose data,
an intensity based on the blood glucose data, and
an intermittence based on the blood glucose data.

20. The annunciator as recited in claim 15 further comprising an ambient light sensor configured to provide a signal indicating an ambient light level, an intensity of the visible indicium being at least partially based on the ambient light level.
